(19) United States
(12) Patent Application Publication
(10) Pub. No.: US 2026/0314964 A1
(43) Pub. Date: Oct. 15, 2026

(54) WEARABLE DEVICE FOR BLOOD OXIMETRY

(71) Applicant: Polar Electro Oy, Kempele (FI)

(72) Inventor: Seppo Korkala, Kempele (FI)

(73) Assignee: Polar Electro Oy, Kempele (FI)

(21) Appl. No.: 19/481,911

(22) PCT Filed: May 23, 2024
(86) PCT No.: PCT/FI2024/050261

§ 371 Date: Nov. 6, 2025

(30) Foreign Application Priority Data
May 24, 2023 (GB) 2307754.8

(51) Int. Cl. A61B 5/1455; A61B 5/00; A61B 5/0205; A61B 5/021; A61B 5/024; A61B 5/318

(52) U.S. Cl. CPC A61B 5/14552; A61B 5/0205; A61B 5/318; A61B 5/742; A61B 5/7475; A61B 5/02125; A61B 5/02427

(57) ABSTRACT

A wearable device for blood oximetry measurement includes a push button, a blood oximetry sensor head, and a processing unit. The blood oximetry sensor head includes a detector and at least two light-emitting diodes, wherein at least the detector is arranged in the push button. The processing unit is configured to detect depression of the push button and to enable, in response to the detected depression, performing a first measurement by the blood oximetry sensor head, wherein the first measurement is a blood oximetry measurement.

### CROSS-REFERENCE TO RELATED APPLICATIONS

This application is a national phase application of International Application No. PCT/FI2024/050261, filed May 23, 2024, which claims benefit and priority to Great Britain Application No. 2307754.8, filed May 24, 2023, which are incorporated by reference herein in their entireties.

### BACKGROUND

### Technical Field

The present invention relates to a field of measuring oxygen saturation in blood.

### SUMMARY

Blood oximetry (peripheral oxygen saturation, SpO₂) is measured by wearable devices from skin areas such as wrist or arm which may be sub-optimal in terms of measurement accuracy. Arteries are located deep under skin surface in the wrist or the arm and changes in the colour of the arterial blood are subtle. Therefore, a measurement arrangement resulting in a stronger SpO₂ signal would be beneficial.

According to an aspect, there is provided the subject-matter of the independent claims. Embodiments are defined in the dependent claims.

### BRIEF DESCRIPTION OF THE DRAWINGS

In the following, various example embodiments will be described in greater detail with reference to the accompanying drawings, in which

FIG. 1 shows a simplified use case scenario and a schematic block diagram;

FIGS. 2 to 6 illustrate exemplary embodiments;

FIGS. 7 and 8 are flow charts illustrating example functionalities; and

FIG. 9 is a signalling diagram illustrating an example functionality.

### DETAILED DESCRIPTION

The following embodiments are exemplary. Although the specification may refer to “an”, “one”, or “some” embodiment(s) in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments/examples to consist of only those features that have been mentioned and such embodiments may contain also features/structures that have not been specifically mentioned. Further, although terms including ordinal numbers, such as “first”, “second”, etc., may be used for describing various elements, the structural elements are not restricted by the terms. The terms are used merely for the purpose of distinguishing an element from other elements. For example, a first element could be termed a second element, and similarly, a second element could be also termed a first element without departing from the scope of the present disclosure.

Different embodiments and examples are described below using single units, models, equipment, and memory, without restricting the embodiments/examples to such a solution. Concepts called cloud computing and/or virtualization may be used. The virtualization may allow a single physical computing device to host one or more instances of virtual machines that appear and operate as independent computing devices, so that a single physical computing device can create, maintain, delete, or otherwise manage virtual machines in a dynamic manner. It is also possible that device operations will be distributed among a plurality of servers, nodes, devices, or hosts. In cloud computing network devices, computing devices, and/or storage devices provide shared resources. Some other technology advancements, such as Software-Defined Networking (SDN), may cause one or more of the functionalities described below to be migrated to any corresponding abstraction or apparatus or device. Correspondingly, Web 3.0, also known as the third-generation internet, implementing for example blockchain technology, may cause one or more of the functionalities described below to be distributed across a plurality of apparatuses or devices. Therefore, all words and expressions should be interpreted broadly, and they are intended to illustrate, not to restrict, the embodiment.

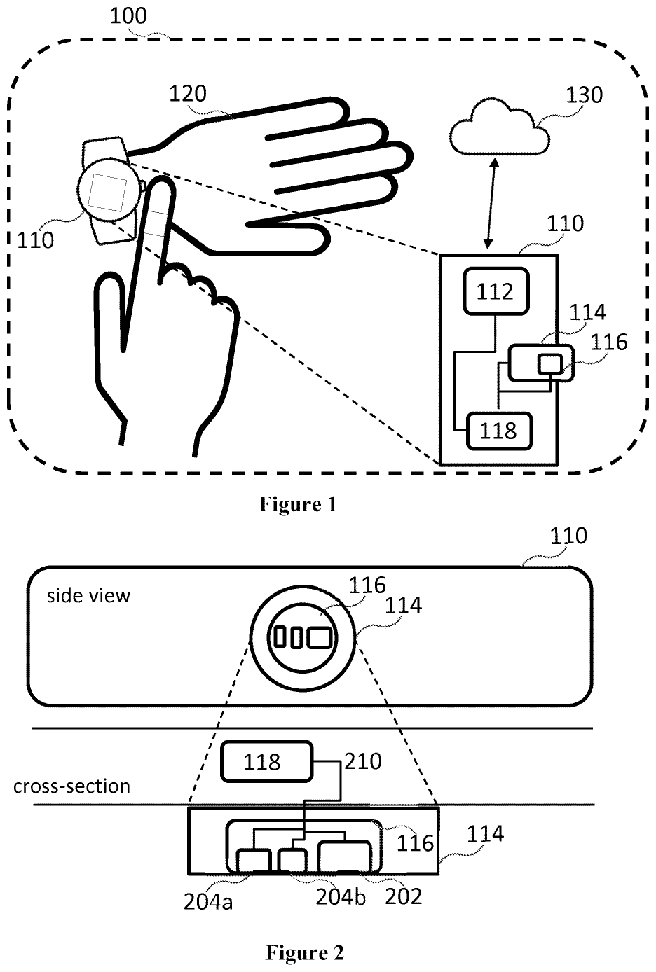

A simplified use case scenario of a wearable device configured to perform blood oximetry is illustrated in FIG. 1. FIG. 1 is a schematic block diagram showing only some functional entities, all being logical units whose implementation and/or number may differ from what is shown. The connections shown in FIG. 1 are logical connections; the actual physical connections may be different. It is apparent to a person skilled in the art that the wearable device comprises any number of shown elements, other equipment, other functions, and other structures that are not illustrated. They, as well as the protocols used, are well known by persons skilled in the art and are irrelevant to the actual invention. Therefore, they need not to be discussed in more detail here.

In the example illustrated in FIG. 1, the use case scenario 100 comprises a wearable device 110 wearable by a user 120 against skin and optionally one or more clouds 130. The wearable device may be, e.g., a wrist device such as a smart watch or a wrist band, or a device wearable on arm. The wearable device 110 comprises at least a push button 114, a blood oximetry sensor head 116, and a processing unit 118. The wearable device may comprise a display unit 112. The wearable device 110 may be connectable to the one or more clouds 130 via one or more networks (not shown in FIG. 1). The wearable device 110 may transmit information such as measurement results to the one or more clouds 130.

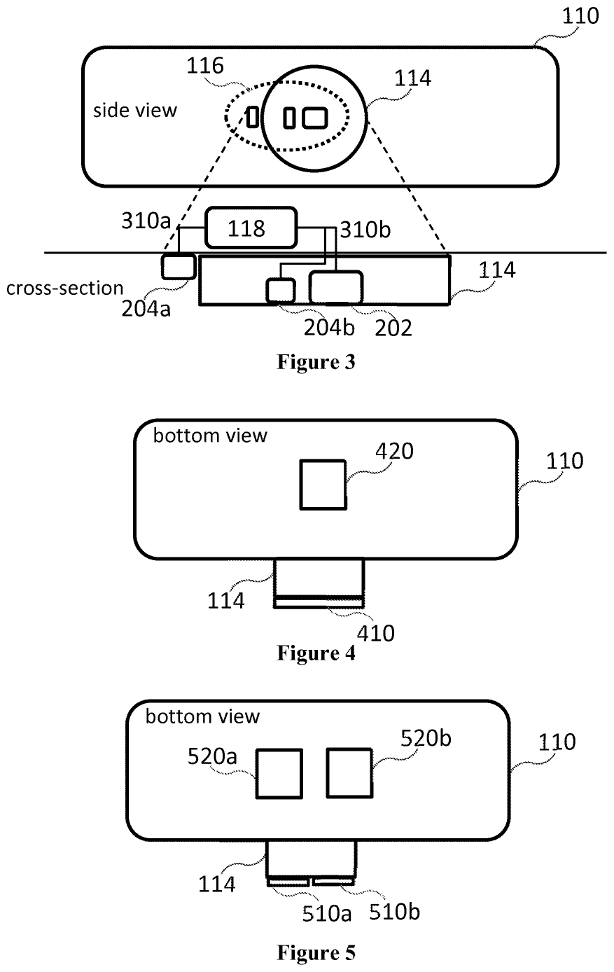

The blood oximetry sensor head 116 comprises at least one detector (a photodiode) and at least two light-emitting diodes (LEDs), wherein at least one LED emits infrared or near-infrared light and at least another LED emits visible red light. Visible red light and (near-)infrared light are absorbed differently by the user's blood. Oxygen saturation of the user's blood can be determined by measuring light signals that have travelled inside the user's finger and then computing a ratio of measured (near-)infrared light signals to measured red light signals. The computed ratio may be expressed as a percentage. Wavelength of the (near-)infrared light emitted by the blood oximetry sensor head may be, e.g., 940 nm (nanometres). Wavelength of the red light emitted by the blood oximetry sensor head may be, e.g., 660 nm and/or 530 nm. Arteries in the finger are closer to skin surface than arteries, e.g., in a wrist area or an arm area. Therefore, performing blood oximetry measurement on the finger results in a stronger signal even without attaching a clipping device such as a pulse oximeter to the finger. The at least one detector is arranged in the push button, e.g., at a centre of the push button. The at least two light-emitting diodes may be arranged in the push button 114 or near the push button 114 as described below with reference to FIGS. 2 and 3. Near the push button may be understood herein as, e.g., touching an edge of the push button or being located in an immediate vicinity of the push button. Arranging at least one of the at least two light-emitting diodes near the push button may result in achieving a greater area of illumination for the blood oximetry measurement.

The processing unit 118 is configured at least to enable performing a blood oximetry measurement as explained in more detail below. The processing unit 118 may comprise at least one processor and at least one memory storing a computer program comprising program instructions that configure the at least one processor to execute, e.g., embodiments described herein. The memory may further store a configuration database defining parameters for the processor(s). The processing unit 118 is coupled to the push button 114 and the blood oximetry sensor head 116 by, e.g., galvanic connections. The at least two light-emitting diodes and the at least one detector may each have a separate galvanic connection to the processing unit 118.

The display unit 112 may be configured to display information and instructions to the user. The display unit may be, e.g., a screen. The wearable device 110 may further comprise a user input unit (not shown in FIG. 1) such as a touch user interface.

The wearable device 110 may further comprise a communication unit (not shown in FIG. 1) configured to transmit information and measurement data acquired by the wearable device to an external device such as a smart phone or a personal computer or to the one or more clouds 130. The communication unit may be a wireless communication unit supporting a wireless communication protocol such as ANT, ANT+, or Bluetooth®, e.g., Bluetooth Smart®.

FIG. 2 is a simplified block diagram illustrating a schematic structure of an embodiment of the wearable device 110. The push button 114 may be comprised, e.g., on a side of the wearable device 110 or in another location in the wearable device 110 enabling the user to touch or press the push button 114 with a finger. In an embodiment, the push button 114 is a selection button.

The blood oximetry sensor head 116 comprises at least one detector 202 which may be a photodiode and at least two light-emitting diodes (LEDs) 204a, 204b. The at least one detector 202 is arranged in the push button 114, e.g., at a centre of the push button. In the example illustrated in FIG. 2, the at least two light-emitting diodes 204a, 204b are arranged in the push button 114. The at least one detector 202 and the at least two light-emitting diodes 204a, 204b are connected to the processing unit 118 via at least one connection 210, which may be, e.g., a galvanic connection. The at least one connection 210 may comprise separate connections for the at least one detector 202 and the at least two light-emitting diodes 204a, 204b.

FIG. 3 is a simplified block diagram illustrating a schematic structure of an embodiment of the wearable device 110. In the embodiment illustrated in FIG. 3, at least one light-emitting diode 204a of the at least two light-emitting diodes 204a, 204b is arranged near the push button 114. In an embodiment, the at least two light-emitting diodes 204a, 204b are arranged near the push button 114. Arranging at least one of the at least two light-emitting diodes near the push button enables illuminating a larger portion of the finger. The at least one detector 202 and the at least two light-emitting diodes 204a, 204b are connected to the processing unit 118 via connections 310a, 301b, which may be, e.g., galvanic connections.

FIG. 4 is a simplified block diagram illustrating a schematic structure of an embodiment of the wearable device 110 further configured to perform electrocardiogram measurements. At least one skin electrode 410 is arranged on a surface of the push button 114. At least one skin electrode 420 is arranged on a bottom surface of the wearable device 110 wearable against the user's skin. An electrocardiogram measurement may be performed via the at least one skin electrode 410 arranged on the surface of the push button 114 and via the at least one skin electrode 420 arranged on the bottom surface of the wearable device 110 wearable against the user's skin. The at least one skin electrode 420 on the bottom surface measures voltage changes on the user's skin surface. The at least one skin electrode 410 on the surface of the push button measures voltage changes on the user's finger skin surface. The finger is a finger in another hand from the hand wearing the wearable device in order to obtain signals from both atrial and ventrical sides of the heart. The processing unit 118 is coupled to the at least one skin electrode 410 on the surface of the push button 114 and the at least one skin electrode 420 on the bottom surface of the wearable device 110 by, e.g., a galvanic connection. The at least one skin electrode 410 on the surface of the push button 114 and the at least one skin electrode 420 on the bottom surface of the wearable device 110 may each have a separate galvanic connection to the processing unit 118.

In an embodiment, the at least one skin electrode 410 on the surface of the push button is a ring that is arranged such that it surrounds the detector 202 of the blood oximetry sensor head, that is, there is an opening within the at least one skin electrode 410 for the detector 202.

FIG. 5 is a simplified block diagram illustrating a schematic structure of an embodiment of the wearable device 110 further configured to perform a bioimpedance measurement. At least two skin electrodes 510a, 510b are arranged on the surface of the push button 114. At least two skin electrodes 520a, 520b are arranged on the bottom surface of the wearable device 110 wearable against the user's skin. The at least two skin electrodes 510a, 510b on the surface of the push button and the at least two skin electrodes 520a, 520b on the bottom surface of the wearable device comprise current electrodes configured at least to drive electricity into the subject (user) and voltage electrodes configured at least to measure voltage generated in the subject. The electricity driven into the subject may be a low alternating electric current on one or more frequencies. The processing unit 118 is coupled to the at least two skin electrodes 510a, 510b on the surface of the push button 114 and the at least two skin electrodes 520a, 520b on the bottom surface of the wearable device 110 by, e.g., a galvanic connection. The at least two skin electrodes 510a, 510b on the surface of the push button 114 and the at least two skin electrodes 520a, 520b on the bottom surface of the wearable device 110 may each have a separate galvanic connection to the processing unit 118.

FIG. 6 is a simplified block diagram illustrating a schematic structure of an embodiment of the wearable device further configured to perform a heart pulse detection timing measurement. The push button 114 comprises the blood oximetry sensor head 116. A photoplethysmography sensor head 610 is arranged in the push button 114. The photoplethysmography sensor head 610 comprises at least one light-emitting diode and at least one photodiode (a light detector). The at least one light-emitting diode illuminates the user's skin and the at least one photodiode measures intensity of light reflecting and scattering from the user's skin. The blood oximetry sensor head 116 and the photoplethysmography sensor head 610 are connected to the processing unit 118 via at least one connection 620, which may be, e.g., a galvanic connection. The at least one connection 620 may comprise separate connections for blood oximetry sensor head 116 and the photoplethysmography sensor head 610.

In an embodiment, the heart pulse detection timing measurement is a pulse transit time measurement. The pulse transit time measurement is a measurement of a time a heart pulse wave travels through a user's body. The processing unit is further configured to determine blood pressure on the basis of the pulse transit time measurement.

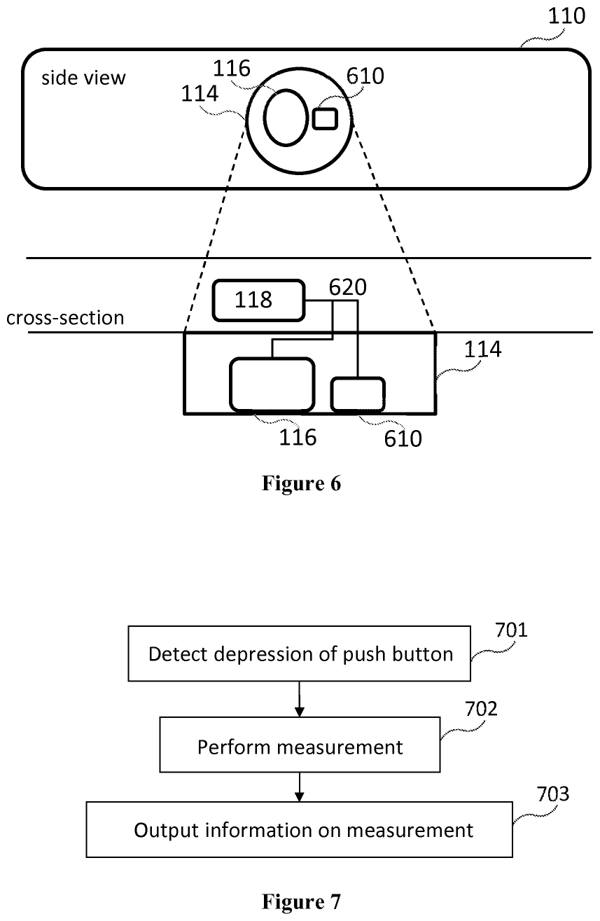

Exemplary functionalities of the processing unit 118 are illustrated in FIG. 7. A measurement may be started by the user of the wearable device by pushing or pressing the push button of the wearable device with a finger. The measurement may be a first measurement. A depression of the push button is detected in block 701. In response to the detection, performing the measurement by the blood oximetry sensor head is enabled in block 702. The measurement is a blood oximetry measurement. The performing the measurement may last, e.g., 20 or 30 seconds. The user is required to keep the finger on the push button or press the push button with the finger during the measurement. Outputting information on the measurement through the display unit is enabled in block 703. In an embodiment, the processing unit may determine a training guidance instruction on the basis of blood oximetry measurement results and output the training guidance instruction to the user through the display unit. For example, if a result of the blood oximetry measurement is below a pre-determined threshold, the processing unit may be configured to remove at least one exercise planned for the user for today or to reduce an intensity level of the at least one exercise.

The processing unit is further configured to terminate the measurement when the measurement is completed or the depression of the push button by the finger is stopped. The measurement may be understood herein to be completed, e.g., when at least one result of the measurement, such as a parameter, is obtained by the processing unit. The outputting the information on the measurement may be enabled after the processing unit has terminated the measurement. The information may comprise measurement results, e.g., at least one parameter of the blood oximetry measurement. The at least one parameter may be a percentage, such as 95% or 98%. The processing unit may be further configured to store the measurement results in a user account of the user of the wearable device.

In an embodiment, the outputting the information on the measurement is enabled during performing the measurement. The information may further comprise, e.g., an instruction displayed to the user during the measurement. The instruction may guide the user to conduct the measurement, e.g., by instructing the user to keep the finger on the push button until the measurement is completed. The instruction may comprise, e.g., a timer, a light, or an image indicating how long the user's finger is recommended to be kept on the push button.

In an embodiment, the processing unit may be further configured to enable performing a second measurement and outputting information on the second measurement through the display unit.

In an embodiment, the second measurement may be an electrocardiogram measurement. The electrocardiogram measurement may be performed as described above with reference to FIG. 4 via the at least one skin electrode 410 arranged on the surface of the push button 114 and the at least one skin electrode 420 arranged on the bottom surface of the wearable device 110 wearable against a user's skin.

In an embodiment, the second measurement may be a bioimpedance measurement. The bioimpedance measurement may be performed as described above with reference to FIG. 5 via the at least two skin electrodes 510a, 510b on the surface of the push button 114 and the at least two skin electrodes 520a, 520b on the bottom surface of the wearable device 110 wearable against the user's skin.

In an embodiment, the processing unit may be further configured to enable performing a third measurement and outputting information on the third measurement through the display unit.

In an embodiment, the third measurement may be a heart pulse detection timing measurement. The heart pulse detection timing measurement may be performed as described above with reference to FIG. 6 via the photoplethysmography sensor head 610 arranged in push button 114. In an embodiment, the heart pulse detection timing measurement may be a pulse transit time measurement.

In an embodiment, the wearable device comprises the at least one skin electrode arranged on the surface of the push button and the at least one skin electrode arranged on the bottom surface of the wearable device wearable against a user's skin as described above with reference to FIG. 4, and the photoplethysmography sensor head arranged in the push button as described above with reference to FIG. 6. The second measurement is the electrocardiogram measurement and the third measurement is the pulse transit time measurement. The processing unit is further configured to determine blood pressure on the basis of the pulse transit time measurement and then to determine the blood pressure further on the basis of the electrocardiogram measurement.

Exemplary further functionalities of the processing unit 118 are illustrated in FIG. 8. In an embodiment, the push button is a selection button of the wearable device comprising a context-sensitive selection function. A depression of the push button is detected in block 801. The depression of the push button is realised by a finger of the user. A context of the wearable device wherein the detected depression occurs is determined in block 802. The context of the wearable device may be understood, e.g., as a mode of the wearable device, such as a training mode, a measurement mode, or a rest mode. The user may select the context of the wearable device by pressing the push button one or more times. The context of the wearable device may be displayed to the user through the display unit, e.g., when the depression of the push button is detected. If the detected depression occurs in a first context of the wearable device (block 802: yes), the blood oximetry sensor head is enabled in block 803. If the detected depression occurs in a second context of the wearable device and not in the first context (block 802: no), the blood oximetry sensor head is disabled in block 804. The disabling the blood oximetry sensor head may be understood herein as not enabling the blood oximetry sensor head. When the blood oximetry sensor is disabled, other measurements may be performed by the wearable device. The other measurements may comprise the second measurement, which may be the electrocardiogram measurement or the bioimpedance measurement, and/or the third measurement, as described in more detail above.

In an embodiment, the wearable device may output health information to the user through the display unit based on the measurement results. The health information may comprise health or activity recommendations. In an embodiment, the wearable device may transmit a notification, e.g., to an elderly care service, based on the measurement results.

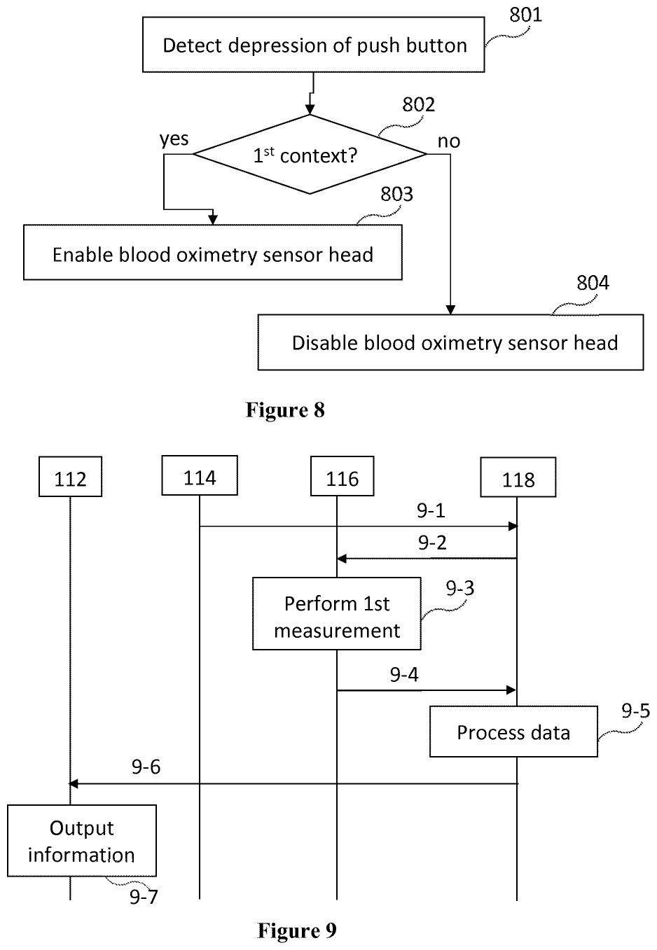

FIG. 9 illustrates a signalling diagram according to an embodiment of internal information exchange within the wearable device. The processing unit 118 detects (message 9-1) a depression of the push button 114. Upon detecting said depression, the processing unit 118 enables (message 9-2) performing a first measurement by the blood oximetry sensor head 116. The first measurement is performed in block 9-3 by the blood oximetry sensor head 116. The blood oximetry sensor head 116 transmits (message 9-4) data of the first measurement to the processing unit 118, which processes in block 9-5 the data. The processing unit enables (message 9-6) outputting information on the first measurement through the display unit 112. The information is outputted in block 9-7 by the display unit 112. The data (message 9-4) and the enabling the outputting of the information (message 9-6) may also occur multiple times during the process or continuously during the process.

In an embodiment, the processing unit or a portion of the processing unit may be located outside the wearable device. Thus, some of the functionalities illustrated in FIG. 9 may occur also outside the wearable device.

The embodiments described herein are not medical devices. The measurements performed may provide indicative information not intended to be used for diagnostic or medical purposes.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), graphics processing units (GPUs), processors, controllers, micro-controllers, microprocessors, other electronic units designed to perform the functions described herein, or a combination thereof. For firmware or software, the implementation can be carried out through modules of at least one chipset (e.g., procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art. It will be obvious to a person skilled in the art that, as the technology advances, the inventive concept can be implemented in various ways. The invention and its embodiments are not limited to the examples described above but may vary within the scope of the claims.

What is claimed is:

1. A wearable device comprising:
a push button;
a blood oximetry sensor head comprising a detector and at least two light-emitting diodes, wherein at least the detector is arranged in the push button;
at least one skin electrode on a surface of the push button and one skin electrode on a bottom surface of the wearable device wearable against a user's skin; and
a processing unit configured to detect a depression of the push button and to enable, in response to the detected depression, performing a first measurement by the blood oximetry sensor head, wherein the first measurement is a blood oximetry measurement, wherein the processing unit is further configured to enable, in response to the detected depression, performing a second measurement via the at least one skin electrode on the surface of the push button and the at least one skin electrode on the bottom surface of the wearable device.

2. The wearable device of claim 1, further comprising a display unit, wherein the processing unit is further configured to enable outputting information on the first measurement through the display unit.

3. The wearable device of claim 2, wherein the processing unit is configured to enable the outputting the information during the performing of the first measurement.

4. The wearable device of claim 1, wherein the at least two light-emitting diodes are arranged in the push button.

5. The wearable device of claim 1, wherein at least one of the at least two light-emitting diodes is arranged near the push button.

6. The wearable device of claim 1, wherein the detector is arranged at a centre of the push button.

7. The wearable device of claim 1, wherein the second measurement is an electrocardiogram measurement.

8. The wearable device of claim 1, wherein the wearable device comprises at least two skin electrodes on the surface of the push button and at least two skin electrodes on the bottom surface of the wearable device, and wherein the second measurement is a bioimpedance measurement.

9. The wearable device of claim 1, wherein the wearable device further comprises a photoplethysmography sensor head arranged in the push button, and wherein the processing unit is further configured to enable, in response to the detected depression, performing a third measurement by the photoplethysmography sensor head, wherein the third measurement is a heart pulse detection timing measurement.

10. The wearable device of claim 9, wherein the pulse detection timing measurement is a pulse transit time measurement, and the processing unit is further configured to determine, on the basis of the pulse transit time measurement, blood pressure.

11. The wearable device of claim 7, wherein the wearable device further comprises a photoplethysmography sensor head arranged in the push button, wherein the processing unit is further configured to enable, in response to the detected depression, performing a third measurement by the photoplethysmography sensor head, wherein the third measurement is a pulse transit time measurement, and wherein the processing unit is further configured to determine, on the basis of the pulse transit time measurement, blood pressure and to determine, on the basis of the electrocardiogram measurement, the blood pressure further.

12. The wearable device of claim 2, wherein the processing unit is further configured to trigger, in response to the detected depression of the push button, measurements comprising at least the first measurement, and wherein the information on said measurements comprises at least one instruction, to a user of the wearable device, to conduct said measurements and at least one parameter measured during said measurements.

13. The wearable device of claim 1, wherein the push button is a selection button of the wearable device comprising a context-sensitive selection function, and wherein the processing unit is configured to enable, in response to the detected depression occurring in a first context of the wearable device, the blood oximetry sensor head and disable, in response to the detected depression occurring in a second context of the wearable device, the blood oximetry sensor head.

14. The wearable device of claim 1, wherein the processing unit is further configured to terminate the measurement when the measurement is completed or the depression of the push button by the finger is stopped.

15. A method comprising:
detecting a depression of a push button of a wearable device; and
enabling, in response to the detected depression, performance of a first measurement by a blood oximetry sensor head comprising a detector and at least two light-emitting diodes, wherein at least the detector is arranged in the push button, and wherein the first measurement is a blood oximetry measurement; and
enabling further, in response to the detected depression, performance of a second measurement via at least one skin electrode on a surface of the push button and at least one skin electrode on a bottom surface of the wearable device wearable against a user's skin.

16. The method of claim 15, further comprising enabling, in response to the detected depression, output of information on the first measurement through a display unit.

17. The method of claim 16, further comprising enabling the outputting the information during the performing the first measurement.

18. A processing unit for a wearable device, the processing unit comprising at least one processor and at least one memory storing a computer program comprising program instructions that, when executed by the at least one processor, cause the processing unit to perform operations comprising:
detecting a depression of a push button of the wearable device;
enabling, in response to the detected depression, performance of a first measurement by a blood oximetry sensor head comprising a detector and at least two light-emitting diodes, wherein at least the detector is arranged in the push button, and wherein the first measurement is a blood oximetry measurement; and
enabling further, in response to the detected depression, performance of a second measurement via at least one skin electrode on a surface of the push button and at least one skin electrode on a bottom surface of the wearable device wearable against a user's skin.

19. The processing unit of claim 18, wherein the program instructions, when executed by the at least one processor, further cause the processing unit to perform operation comprising enabling, in response to the detected depression, the output of information on the first measurement through a display unit.

20. The processing unit of claim 19, wherein the program instructions, when executed by the at least one processor, further cause the processing unit to perform operations comprising enabling the output of the information during performance of the first measurement.
